(19) United States
(12) Patent Application Publication
(10) Pub. No.: US 2013/0096202 A1
(43) Pub. Date: Apr. 18, 2013

(54) DISINFECTING AND STERILISING SOLUTIONS

(71) Applicant: John Toft, Stockport (GB)

(72) Inventor: John Toft, Stockport (GB)

(73) Assignee: GX LABS HOLDINGS LIMITED, London (GB)

(21) Appl. No.: 13/806,422

(22) PCT Filed: Jun. 24, 2011
(86) PCT No.: PCT/GB11/51198

§ 371 Date: Dec. 21, 2012

(30) Foreign Application Priority Data
Jun. 25, 2010 (GB) 1010717.5

(51) Int. Cl. A61K 47/46

(52) U.S. Cl. CPC A61K 47/46
USPC 514/705

(57) ABSTRACT

The present invention relates to disinfecting and sterilising solutions containing glutaraldehyde. In particular, the invention provides solutions which are stabilised through the use of a unique combination of additives, including a bioflavonoid antioxidant (or other phenolic antioxidant) and an alkyl polyglucoside surfactant.

### BACKGROUND

The sanitisation, disinfection and sterilising properties of glutaraldehyde are well documented in the prior art.

Both alkaline and acid solutions of glutaraldehyde are known to lose their biocidal activity during extended shelf storage. Room temperature stability studies of commercial acid glutaraldehyde (2%) demonstrate a maximum loss in the active ingredient of the order of 7 per cent after 24 months. In contrast, room temperature ageing studies of alkaline glutaraldehyde (2% Cidex-7) demonstrated losses of 14 to 18 per cent of the active ingredient one month after buffering (e.g. with sodium bicarbonate).

It is thought that the monomer form of glutaraldehyde (OHC—CH₂—CH₂—CH₂—CHO) is the primary biocidal agent (i.e. in aqueous solutions). The biocidal efficacy of any aldehyde-containing composition is directly related to the number of monomer molecules present at the time the solution is used (the monomer being in equilibrium with the different types of polymeric form). It is therefore postulated that glutaraldehyde disinfectants lose their potency due to three main types of reactions: aldol condensation, hydration-polymerization, and oxidation. The relative magnitude of each depends upon many variables, such as pH, dissolved gases content, and temperature.

In 1972, R. M. G. Boucher et al (Proc. West. Pharmacol. Soc. 16:282-288, 1973) provided a theoretical explanation for the influence of pH on the microbiocidal properties of aqueous glutaraldehyde compositions. An aqueous glutaraldehyde composition contains a small amount of pure aldehyde monomer molecules. The monomer is always present in equilibrium with larger, more complex hydrate molecules (see R. M. G. Boucher, Respiratory Care, Nov. 78, Vol. 23, No. 11, 1063-1071). Hydrates result from the condensation or polymerisation of the small monomers into larger agglomerates. In any aldehyde solution, in the acid form, equilibrium is quickly established between the small monomers and the large polymers/oligomers.

The formation of condensation type polymers is irreversible and the polymers cannot return to the active monomer form even by heating or ultrasonics. Allowing aqueous glutaraldehyde to stand in the alkaline pH range greatly accelerates the rate of polymerisation.

Glutaraldehyde monomers, to the contrary, have a slower rate of polymerisation in the acid range (see Rasmussen K E et al, Histochemistry, 38:19, 1974). Furthermore, acid glutaraldehyde compositions comprise glutaraldehyde monomers in equilibrium with aldol addition type polymers. The formation of the addition type polymers is reversible.

Accordingly, acid glutaraldehyde solutions have a far longer biocidal shelf life than alkaline solutions.

It is thought that the biocidal activity of glutaraldehyde results from its capacity to react with and thereby cross-link amine groups expressed on various pathogens. This is supported by the work of T. J. Munton, J. Appl. Bact., 33:410-619, 1970 and D. Hopwood, Histochemie, 17:151, 1968. The reactive amine species is —NH₂ and not —NH₃⁺. The biocidal activity of glutaraldehyde is therefore pH dependent and glutaraldehyde has its greatest biocidal activity in the neutral to alkaline pH range. Of course, this is the region in which glutaraldehyde most unstable as explained above.

It is considered that the optimum pH for biocidal activity and stability is therefore around pH 5-7.

Glutaraldehyde, at normal use concentrations, has been reported by some hospital personnel to have a pungent odour and be irritating to the eyes and nasal passages and glycol has been added to formulations to counteract this, as detailed in the prior art summary below.

U.S. Pat. No. 3,016,328 discloses chemical sterilization compositions comprising a mixture of a saturated dialdehyde (including glutaraldehyde), a lower alkanol containing from 1 to 3 carbon atoms and an alkali metal carbonate or bicarbonate alkalinating agent. The lower alkanol component is disclosed as being selected from isopropanol, methanol, ethanol and n-propanol. This document emphases the importance of including an alkaline additive in order for the sterilizing composition to have the desired spore killing activity. For example, it is disclosed that it is necessary that the solution has a pH value in excess of 7.4 in the final solution.

U.S. Pat. No. 3,282,775 discloses a sterilization composition comprising a saturated dialdehyde (including glutaraldehyde) and a cationic surfactant. The compositions may be used over a wide pH range, i.e. a pH of from 4.0 to 9.0. However, it is well known that cationic surfactants are toxic and have allergenic and irritant properties. The exemplified cationic surfactant, cetylpyridinium chloride, has very high levels of toxicity when inhaled.

U.S. Pat. No. 4,093,744 discloses a composition comprising glutaraldehyde and a detergent selected from non-ionic, anionic and ampholytic surfactants. The compositions are disclosed as having a pH no greater than 7.4, and preferably 6.5 to 7.4. It is disclosed that these compositions are effective in killing various bacterial spores including Clostridium welchii (Cl. welchii), Clostridium tetani (Cl. tetani), Bacillus subtilis (B. subtilis), Bacillus pumilus (B. pumilus), Bacillus globigii (B. globigii) and Clostridium sporogenes (Cl. sporogenes).

U.S. Pat. No. 3,983,252 discloses a composition comprising glutaraldehyde and an alkali metal salt of a hydrocarbon carboxylic acid, e.g. sodium citrate or sodium acetate. The composition is disclosed as having a pH in the range of 6 to 8. It is also disclosed that the stability of the composition can be enhanced by the inclusion of an alcohol, a diol or a triol.

U.S. Pat. No. 3,968,250 discloses a composition comprising glutaraldehyde and a non-ionic surfactants or an anionic surfactant. An example of the surfactant employed in the composition is an alcohol ethoxylate. It is disclosed that the non-ionic surfactants and anionic surfactants are more active than cationic surfactants.

FR 2,321,300 discloses that quaternary ammonium compounds have an effect on pathogens in the food industry.

U.S. Pat. No. 4,208,404 discloses a composition comprising glutaraldehyde and a highly ionisable salt. It is disclosed that the composition does not require a surfactant. Examples of the highly ionisable salts include neutral lithium, sodium, potassium and ammonium salts of the inorganic acids, sulphuric acid, hydrochloric acid or nitric acid or the organic acids, citric acid or acetic acid.

U.S. Pat. No. 4,923,899 discloses a composition comprising glutaraldehyde, a quaternary ammonium compound and an aliphatic hydroxyl compound. An essential feature of this composition is the positively charged, non-metallic nuclei. An optional component of the composition is a chelating agent which is employed to aid the solubility of the other components and to counteract any deleterious effects from diluting concentrated commercial strengths with hard water. Additionally, the chelating agent can help to break down the coatings of spores. The preferred chelating agents disclosed are ethylene diamine tetra acetic acid (EDTA) and partial esters or salts of EDTA, such as tetrasodium ethylenediamine tetraacetate. As mentioned above, it is well known that cationic surfactants, such as the quaternary ammonium compounds, are toxic and have allergenic and irritant properties. The exemplified cationic surfactant, cetyldimethylethylammonium bromide, is irritating to skin, can cause serious damage to eyes, and causes stomach pain, vomiting, diarrhoea and convulsions if swallowed.

U.S. Pat. No. 5,219,890 discloses a composition comprising glutaraldehyde and anionic surfactants of the alkyl sulphate, alkyl sulphonate, alcohol sulphate or alkyl aryl sulphonate type. It is disclosed that the anionic surfactants are superior to non-ionic surfactants.

U.S. Pat. No. 4,748,279 discloses a three component composition comprising glutaraldehyde , a non-ionic surfactant and a glycol. The non-ionic surfactant is based upon ethylene oxide, specifically alcohol ethoxylates.

US 2001/0009682 discloses a composition comprising glutaraldehyde, a glycol or polyol and a lithium based buffer capable of maintaining the concentrate at a pH of 6 or above. The composition may additionally contain an anionic or non-ionic surfactant.

It is an aim of the present invention to provide a glutaraldehyde composition for disinfecting and/or sterilising a substrate, which has a broad spectrum of biocidal activity. It is therefore an aim of the present invention to provide a glutaraldehyde composition for disinfecting and/or sterilising a substrate which has activity against bacteria, mycobacteria, spores, fungi and viruses. It is another aim of the present invention to provide a glutaraldehyde composition for disinfecting and/or sterilising a substrate having an activity equal to or better than conventional glutaraldehyde compositions. It is another aim of the present invention to provide a composition for disinfecting and/or sterilising a substrate which is fast acting.

It is another aim of the present invention to provide a disinfecting and sterilising glutaraldehyde composition which is safe to use at low concentrations. It is another aim of the present invention to provide a disinfecting and sterilising glutaraldehyde composition which is environmentally friendly and utilises natural occurring products. It is another aim of the present invention to provide a disinfecting and sterilising glutaraldehyde composition which is non corrosive to hard surfaces. It is another aim of the present invention to provide a disinfecting and sterilising glutaraldehyde composition which has a low odour. It is another aim of the present invention to provide a disinfecting and sterilising glutaraldehyde composition which operates at a neutral pH. It is another aim of the present invention to provide a disinfecting and sterilising glutaraldehyde composition which avoids problems with precipitation from hard water.

The compositions of the present invention seek to address all or some of the above aims.

The compositions disclosed herein find application in a variety of settings, including in hospitals, e.g. for disinfecting and sterilising endoscopes and other medical and surgical instruments; in health care and domestic setting, e.g. for hand and skin washes; and in animal enclosures, e.g. for animal skin washes.

### SUMMARY OF THE INVENTION

According to a first aspect, the present invention provides a disinfecting and sterilising composition comprising:
from 0.05 to 45% (w/w) glutaraldehyde;
an alkyl polyglucoside surfactant;
a buffer effective to maintain the composition in the pH range 5 to 7; and
a bioflavonoid antioxidant or other phenolic antioxidant.

Herein, a “phenolic antioxidant” is a compound comprising a phenol moiety that is capable of exhibiting an antioxidant effect, especially an antioxidant effect capable of stabilising glutaraldehyde. Features, including preferred, suitable, and optional features disclosed herein in relation to the bioflavonoid are also features which apply in relation to the “other phenolic antioxidant”.

Bioflavonoids belong to the family of phenolic antioxidants, also known as phenol oxidative inhibitors.

Phenol oxidative inhibitors, such as 2-phenylbenzopyrane, hydroxyphenylethanol, benzophenone, benzotriazole, hydroxyphenolethanol, propyl trihydroxybenzoate, or a cinnamate compound (or cinnamic acid derivative) (e.g. Tinogard from Ciba) as mentioned above, have proved to be surprisingly effective as auto-oxidative inhibitors for glutaraldehyde over a wide range of concentrations and pH.

In a particular embodiment, the composition comprises a bioflavonoid antioxidant. In a particular embodiment, the composition comprises a bioflavonoid antioxidant and is free of other phenolic antioxidants.

As can be seen from the examples, the composition of the present invention is typically manufactured from a commercially available liquid containing 50% glutaraldehyde. The higher the concentration of glutaraldehyde in the product, the more economical it would be to manufacture, transport and distribute. Therefore, the composition of the invention may be a ‘super concentrate’ and may include up to 45% (w/w) glutaraldehyde. In an embodiment, the composition comprises from 0.05 to 20% (w/w) glutaraldehyde. Preferably, the composition comprises from 0.05 to 15% (w/w) glutaraldehyde. More preferably, the composition comprises from 0.05 to 10% (w/w) glutaraldehyde.

Typically a composition comprising 10% (w/w) or more of glutaraldehyde is used as a concentrate solution which is subsequently diluted for use as the final commercial product, e.g. a hand sanitiser, a hard surface cleaner or a high level disinfectant.

When the end product is a hand sanitiser, the composition will typically comprise from 0.05 to 0.25% (w/w) glutaraldehyde. Therefore, in an embodiment, the composition comprises from 0.05 to 0.25% (w/w) glutaraldehyde. When the end product is a hard surface cleaner, the composition will typically comprise from 0.25 to 0.5% (w/w) glutaraldehyde. Therefore, in an embodiment, the composition comprises from 0.25 to 0.5% (w/w) glutaraldehyde. When the end product is a high level disinfectant, the composition will typically comprise from 0.5 to 5% (w/w) glutaraldehyde. Therefore, in an embodiment, the composition comprises from 0.5 to 5% (w/w) glutaraldehyde.

In an embodiment, the buffer is effective to maintain the composition in the pH range 5.5 to 6.5. Any suitable buffer may be used. In an embodiment, the buffer is selected from the group comprising: phosphate, acetate, citrate, sulfonic acid, ascorbate, linolenate, carbonate and bicarbonate based buffers. In an embodiment, the buffer is selected from the group comprising: phosphate, acetate, citrate, sulfonic acid, carbonate and bicarbonate based buffers. In an embodiment, the buffer is a citric acid and sodium citrate mixture. In an embodiment, the composition comprises from 0.01 to 11 g/l of citric acid, preferably 0.05 to 11 g/l of citric acid. In another embodiment, the composition comprises from 0.1 to 11 g/l of citric acid. In an embodiment, the composition comprises from 0.5 to 140 g/l of sodium citrate. In an embodiment, the composition comprises from 1.4 to 140 g/l of sodium citrate. In an embodiment, the composition comprises from 0.1 to 11 g/l of citric acid and from 1.4 to 140 g/l of sodium citrate.

Surfactants are useful in lowering surface tension, and supposedly forming complexes in a similar manner as glycols with glutaraldehyde. Conventional teaching prefers the use of non-ionic surfactants, especially fatty alcohol ethoxylates. However, the applicant has found that these non-ionic surfactants do not generally possess sufficient emulsification power to have a significant effect upon the highly lipid mycobacteria, or the waxy coating on bacterial and fungal spores.

As mentioned above, one of the aims of the present invention is to provide an environmentally-friendly disinfecting and sterilising composition. The applicant has found that the plant based surfactants, alkyl polyglucosides, are of greater effectiveness than conventionally used surfactants. Not meaning to be bound by theory, it is thought that the alkyl polyglucoside surfactants can behave in a similar manner to glycols in protecting the glutaraldehyde molecule in solution.

In an embodiment, the alkyl polyglucoside surfactant is an alkyl polyglucoside surfactant based on natural fatty alcohols. Alkyl polyglucosides are a new generation of ‘green’ surfactants and are often referred to as polysaccharide or sugar surfactants. In an embodiment, the alkyl polyglucoside surfactant is a C₈-C₁₆ alkyl polyglucoside surfactant, e.g. a C₈, C₉, C₁₀, C₁₁, C₁₂, C₁₃, C₁₄, C₁₅ or C₁₆ alkyl polyglucoside surfactant, or a mixture thereof.

For example, the alkyl polyglucoside surfactant may be obtained from Cognis Corporation, Dusseldorf, Germany from their Glucopon® product range. For example, the alkyl polyglucoside surfactant may be Glucopon® 50 G (a C₁₂-C₁₄ polyglucoside), Glucopon® 215 UP (a C₈-C₁₀ polyglucoside), Glucopon® 225 DK (a C₈-C₁₀ polyglucoside), Glucopon® 425 N/HH (a C₈-C₁₄ polyglucoside), Glucopon® 600 CS UP (a C₁₂-C₁₄ polyglucoside) or Glucopon® 650 EC (a C₈-C₁₄ polyglucoside). In an embodiment, the alkyl polyglucoside surfactant is Glucopon 650®.

In an embodiment, the composition comprises from 0.01 to 5.0% (w/w) alkyl polyglucoside.

Of course the actual amount of alkyl polyglucoside will depend on the end use of the formulation. Thus the glutaraldehyde concentrate contains approximately 2% (w/w) alkyl polyglucoside.

It is known that glutaraldehyde can self polymerise by auto-oxidation. Self polymerisation will reduce the activity of the glutaraldehyde and it is therefore desirable to inhibit the self polymerisation process. The applicant has surprisingly found that this process can be inhibited by the addition of antioxidants based on naturally occurring plant extracts, bioflavonoids. Bioflavonoids demonstrate excellent inhibition of the glutaraldehyde oxidation process and thereby help in the stabilisation process. The applicant has also surprisingly found that this process can be inhibited by the addition of other phenolic antioxidants.

The term ‘bioflavonoid’ actually refers to a class of compounds. Bioflavonoids occur naturally and are often found in the skins of citrus fruits. A bioflavonoid has the general structure:

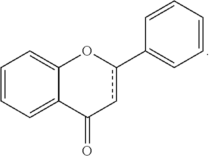

There are four general subgroups of bioflavonoids: flavones, flavonols, flavanones and flavanols. Some examples of bioflavinoids include hesperidin (a glycoside of the flavanone hesperetin), quercitrin and rutin (two glycosides of the flavonol quercetin).

In an embodiment, the composition comprises a flavone bioflavonoid. In an embodiment, the composition comprises a flavonol bioflavonoid. In an embodiment, the composition comprises a flavanone bioflavonoid. In an embodiment, the composition comprises a flavanol bioflavonoid. In an embodiment, the composition comprises from 0.01 to 10% (w/w) bioflavonoid (or other phenolic antioxidant). Of course the actual amount of bioflavonoid will depend on the end use of the formulation. Thus the glutaraldehyde concentrate contains approximately 4% (w/w) bioflavonoid (or other phenolic antioxidant).

The synergism exhibited by the particular combination of bioflavonoid antioxidant and an alkyl polyglucoside surfactant allows a lower amount of glutaraldehyde to be used in the composition than was conventionally possible with the compositions of the prior art. Thus, the compositions of the invention still have activity at concentrations as low as 0.05% glutaraldehyde.

As mentioned previously, glutaraldehyde has a pungent odour, which is reduced by the stabilisation process. The odour can be further reduced by the addition of a deodorising agent, such as a cationic moiety which acts as a deodorising agent. In an embodiment, the composition further comprises soyethyl morpholinium ethosulfate. This compound is believed to loosely interact with the glutaraldehyde molecule and thereby reduce the volatility of glutaraldehyde. This particular deodorising compound is available from Croda Chemicals under the name Forestall®. The amount of deodorising compounds is not essential to the efficacy of the composition of the invention. The pungent odour of the glutaraldehyde can be further masked by the addition of a fragrance. The amount of fragrance added to the composition is not essential to the efficacy of the composition of the invention.

In a particular embodiment, the deodorising agent may be selected from the group including methyl crotonate (e.g. Sinodur™ from Givaudan), Deoplex™ (Saccharomyces ferment by Carrubba Inc.), sodium ricinoleate (obtainable from Chemlink Specialities), zinc ricinoleate (Flexisorb™ by ICT Inc.), cyclodextrin (obtainable from Proctor & Gamble), or a combination thereof.

In a particular embodiment, the deodorising agent comprises methyl crotonate.

The composition of the present invention may also include additional biocidal components. Quaternary ammonium compounds are widely quoted as increasing the fungicidal properties of more dilute versions of glutaraldehyde, and can be included in the range of stabilised glutaraldehyde products. Therefore, the composition of the present invention may also include a cationic biocide such as a quaternary ammonium biocide, e.g. didecyldimethylammonium chloride or derivatives thereof.

The compositions of the present invention may also include additional components to control water hardness and to impart corrosion resistance. In an embodiment, the compositions composition of the invention comprises 0.005-0.1 g/l of a phosphonate sequestrant. Of course the actual amount of the phosphonate sequestrant will depend on the end use of the formulation.

The composition of the present invention may also include additional components to enhance the rate of reaction of glutaraldehyde with spores. For example, highly ionisable salts such as sodium chloride, lithium chloride and sodium acetate may be added to the compositions of the invention to enhance the rate of reaction of the glutaraldehyde with spores.

According to a second aspect, the present invention provides a use of a bioflavonoid antioxidant and an alkyl polyglucoside surfactant to increase the biocidal activity of a glutaraldehyde-containing disinfectant and sterilising composition.

The embodiments of the first aspect apply equally to the second aspect. The disinfecting and sterilising composition may therefore comprise:
from 0.05 to 45% (w/w) glutaraldehyde; and
a buffer effective to maintain the composition in the pH range 5 to 7.

According to a third aspect, the present invention provides a use of a composition of the invention for killing Aspergillus niger.

The invention will now be illustrated by way of the following non-limiting examples in which:

### EXAMPLE 1

### Preparation of 10% Stabilised Glutaraldehyde Concentrate

| Deionised water | 200 | g/l |
|---|---|---|
| Glucopon 650 | 20 | g/l |
| Glutaraldehyde | 200 | g/l |
| Bioflavonoid (Aqualin ECO conc) | 40 | g/l |
| Forestall | 5 | g/l |
| Fragrance | 5 | g/l |
| Citric acid | 5 | g/l |
| Sodium citrate | 60 | g/l |
| NaOH pH modifier | qs | |
| (or adjust with citric acid if too alkaline) | | |
| Sodium Nitrite | 0.05 | g/l |
| Phosphonate sequestrant | 0.005 | g/l |

The glutaraldehyde in the above formulation is a 50% glutaraldehyde solution. For example, the glutaraldehyde can be obtained from Dow Chemical Company under the name “Ucarcide” or from BASF Ludwigshafen Germany under the name “Protectol”.

Forestall® is a deodorising agent available from Croda Chemicals.

In this particular example, the bioflavonoid is commercially available from Stephenson Group as Aqualin ECO conc ®, which contains citrus bioflavonoids, including Neohesperidin, Hesperidin, and Naringin. However, the advantages of the present invention are also apparent when using alternative bioflavonoids, such as green tea extract etc.

All ingredients should be at around 25-30° C. before the method is started.

Firstly, deionised water is added to a mixing vessel. The Glucopon 650 surfactant is then dissolved in the deionised water whilst stirring continuously. The glutaraldehyde, bioflavonoid, and Forestall are then added with mixing. The resulting solution is then allowed to stand for 30 minutes with slow stirring.

The citric acid is then added and the sodium citrate and any other ingredients as required. The pH is then adjusted to pH 5.5-6.5 with 1M NaOH.

This concentrate is used as the starting stabilised glutaraldehyde solution for production of the compositions in the following Examples.

### EXAMPLE 1b

### Preparation of 10% Stabilised Glutaraldehyde Concentrate

| Deionised water | 200 | g/l |
|---|---|---|
| Glucopon 650 | 20 | g/l |
| Glutaraldehyde | 200 | g/l |
| Phenolic antioxidant (Tinogard TS) | 40 | g/l |
| Forestall | 5 | g/l |
| Fragrance | 5 | g/l |
| Citric acid | 5 | g/l |
| Sodium citrate | 60 | g/l |
| NaOH pH modifier | qs | |
| (or adjust with citric acid if too alkaline) | | |
| Sodium Nitrite | 0.05 | g/l |
| Phosphonate sequestrant | 0.005 | g/l |

The glutaraldehyde in the above formulation is a 50% glutaraldehyde solution. For example, the glutaraldehyde can be obtained from Dow Chemical Company under the name “Ucarcide” or from BASF Ludwigshafen Germany under the name “Protectol”.

Forestall® is a deodorising agent available from Croda Chemicals.

In this particular example, the phenolic antioxidant is commercially available from Ciba as Tinogard® TS.

All ingredients should be at around 25-30° C. before the method is started.

Firstly, deionised water is added to a mixing vessel. The Glucopon 650 surfactant is then dissolved in the deionised water whilst stirring continuously. The glutaraldehyde, phenolic antioxidant, and Forestall are then added with mixing. The resulting solution is then allowed to stand for 30 minutes with slow stirring.

The citric acid is then added and the sodium citrate and any other ingredients as required. The pH is then adjusted to pH 5.5-6.5 with 1M NaOH.

This concentrate is used as the starting stabilised glutaraldehyde solution for production of the compositions in Example 5.

### EXAMPLE 2

### High Level Disinfectant

| Deionised or distilled water | 300 | g/l |
|---|---|---|
| Stabilised glutaraldehyde | 300 | g/l |
| (concentrate of Example 1) | (10% strength) | |
| Forestall | 5 | g/l |
| Fragrance | 5 | g/l |
| | (optional) | |
| Defoamer | 1 | g/l |
| Citric acid | 2.5 | g/l |
| Sodium citrate | 30 | g/l |
| pH modifier (NaOH) | qs | |
| Highly ionisable salt | 50 | g/l |
| | (optional) | |
| Bulk with deionised water | 1000 | ml |

All ingredients should be at around 25-30° C. before the method is started.

Firstly, deionised water is added to a mixing vessel. The stabilised glutaraldehyde concentrate is then added, followed by defoamer with mixing. The resulting solution is then allowed to stand for 30 minutes with slow stirring. The citric acid is then added with the sodium citrate and other ingredients with mixing. Finally, the pH is adjusted to pH 5.5-6.5 with 1M NaOH NB. Effective embodiments include compositions comprising the bioflavonoid or other phenolic antioxidant at a concentration between 0.05 to 50grams per litre.

### EXAMPLE 3

### Hard Surface Cleaner

| Deionised or distilled water | 880 | g/l |
|---|---|---|
| Stabilised glutaraldehyde | 30 | g/l |
| (concentrate of Example 1) | (10% strength) | |
| Didecyldimethylammonium chloride | 1 | g/l |
| (and/or benzalkonium chloride) | | |
| Citric acid | 0.5 | g/l |
| Sodium citrate | 7 | g/l |
| pH modifier (NaOH) | qs | |
| Highly ionisable salt | 50 | g/l |
| | (optional) | |
| Bulk with deionised water | 1000 | ml |

All ingredients should be at around 25-30° C. before the method is started.

Firstly, deionised water is added to a mixing vessel. The stabilised glutaraldehyde concentrate is then added. The citric acid and sodium citrate are then added and the pH adjusted to pH 5.5-6.5 with 0.1 M NaOH.

Alternatively, different cationic biocides may be used in place of the didecyldimethylammonium chloride. For example, benzalkonium chloride or biguanide may be used.

### EXAMPLE 4

### Hand Sanitiser

| Deionised or distilled water | 880 | g/l |
|---|---|---|
| Stabilised glutaraldehyde | 10 | g/l |
| (concentrate of Example 1) | (10% strength) | |
| Didecyldimethylammonium chloride | 1 | g/l |
| (and/or benzalkonium chloride) | | |
| Gelling agent | 7 | g/l |
| pH modifier (NaOH) | qs | |
| Bulk with deionised water | 1000 | ml |

All ingredients should be at around 25-30° C. before the method is started.

Firstly, deionised water is added to a mixing vessel. The stabilised glutaraldehyde concentrate is then added, followed by didecyldimethylammonium chloride and then gelling agent. Adjust the pH to 5.5-6.5 with 0.1 M NaOH.

### EXAMPLE 5

### Hard Surface Cleaner

| Deionised or distilled water | 880 | g/l |
|---|---|---|
| Stabilised glutaraldehyde | 30 | g/l |
| (concentrate of Example 1b) | (10% strength) | |
| Didecyldimethylammonium chloride | 1 | g/l |
| (and/or benzalkonium chloride) | | |
| Citric acid | 0.5 | g/l |
| Sodium citrate | 7 | g/l |
| pH modifier (NaOH) | qs | |
| Highly ionisable salt | 50 | g/l |
| | (optional) | |
| Bulk with deionised water | 1000 | ml |

All ingredients should be at around 25-30° C. before the method is started.

Firstly, deionised water is added to a mixing vessel. The stabilised glutaraldehyde concentrate is then added, followed by didecyldimethylammonium chloride and then gelling agent. Adjust the pH to 5.5-6.5 with 0.1 M NaOH.

### EXAMPLE 6

### Results of Trials

The following trials were carried out using the high level disinfectant product of example 2, (that is 2.7% glutaraldehyde concentration). The tests listed in the table below, namely,

EN13727, EN13624, EN14348 and EN14476 are well known to the skilled person and are easily reproducible by following the standard protocols known to the skilled person.

| Sample | | | |
|---|---|---|---|
| Description | Pathogen & Test | Log reduction | Pass/fail status |
| 2.7% stabilised | EN13727 | 5.0 | PASS |
| Glutaraldehyde | (BACTERIA) | | |
| solution | | | |
| 2.7% stabilised | EN13624 | 4.0 | PASS |
| Glutaraldehyde | (FUNGI) | | |
| solution | | | |
| 2.7% stabilised | EN14348 | 5.0 | PASS |
| Glutaraldehyde | (MYCOBACTERIA) | | |
| solution | | | |
| 2.7% stabilised | EN14476 | 4.0 | PASS |
| Glutaraldehyde | (VIRUS) | | |
| solution | | | |

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Compounds, chemical moieties or groups described in conjunction with a particular aspect, embodiment or example of the invention are to be understood to be applicable to any other aspect, embodiment or example described herein unless incompatible therewith.

1-13. (canceled)

14. A disinfecting and sterilising composition comprising:
from 0.05 to 45% (w/w) glutaraldehyde;
an alkyl polyglucoside surfactant;
a buffer effective to maintain the composition in the pH range 5 to 7; and
a bioflavonoid antioxidant or other phenolic antioxidant.

15. The composition of claim 14, wherein the composition comprises from 0.05 to 20% (w/w) glutaraldehyde.

16. The composition of claim 15, wherein the composition comprises from 0.05 to 10% (w/w) glutaraldehyde.

17. The composition of claim 14, wherein the buffer is effective to maintain the composition in the pH range 5.5 to 6.5.

18. The composition of claim 14, wherein the buffer is selected from the group comprising: phosphate, acetate, citrate, sulfonic acid, ascorbate, linolenate, carbonate and bicarbonate based buffers.

19. The composition of claim 14, wherein the composition comprises, as a buffer, from 0.01 to 11g/1 of citric acid and from 0.5 to 140g/1 of sodium citrate.

20. The composition of claim 14, wherein the alkyl polyglucoside surfactant is a C8-C16 alkyl polyglucoside surfactant.

21. The composition of claim 20, wherein the alkyl polyglucoside surfactant is Glucopon® 650 EC.

22. The composition of claim 14, wherein the composition comprises from 0.01 to 5.0% (w/w) alkyl polyglucoside.

23. The composition of claim 14, wherein the composition comprises from 0.01 to 10% (w/w) bioflavonoid.

24. A method of killing Aspergillus niger, comprising:
providing the composition according to claim 14; and
using the composition to kill Aspergillus niger.

25. A method of increasing the biocidal activity of a glutaraldehyde-containing disinfectant and sterilising composition, comprising:
providing a bioflavonoid antioxidant or other phenolic antioxidant and an alkyl polyglucoside surfactant; and
using the bioflavonoid antioxidant or other phenolic antioxidant and the alkyl polyglucoside surfactant to increase the biocidal activity of the glutaraldehyde-containing disinfectant and sterilising composition.

26. The method of claim 25 wherein the disinfecting and sterilising composition comprises:
from 0.05 to 45% (w/w) glutaraldehyde; and
a buffer effective to maintain the composition in the pH range 5 to 7.
